(19) United States
(12) Patent Application Publication
(10) Pub. No.: US 2026/0364880 A1
(43) Pub. Date: Nov. 19, 2026

(54) 3D PRINTING METHOD FOR FORMING DIRECTIONAL FIBER STRUCTURES IN SITU

(71) Applicant: TSINGHUA UNIVERSITY, Beijing (CN)

(72) Inventors: Liliang OUYANG, Beijing (CN); Dezhi ZHOU, Beijing (CN); Bohan DOU, Beijing (CN)

(73) Assignee: TSINGHUA UNIVERSITY, Beijing (CN)

(21) Appl. No.: 19/515,805

(22) PCT Filed: Nov. 23, 2023
(86) PCT No.: PCT/CN2023/133526

§ 371 Date: Mar. 3, 2026

(51) Int. Cl. B29C 64/314; B29C 64/118; B29K 71/00; B29K 105/00; B33Y 10/00; B33Y 40/10; B33Y 40/20; B33Y 70/10; C12N 5/00

(52) U.S. Cl. CPC B29C 64/314; B29C 64/118; B33Y 10/00; B33Y 40/10; B33Y 40/20; B33Y 70/10; C12N 5/0062; B29K 2071/00; B29K 2105/0085; B29K 2489/00; C12N 2533/30; C12N 2533/54; C12N 2537/10; C12N 2539/00

(57) ABSTRACT

A 3D printing method for forming oriented fiber structures in situ is provided. The method includes: preparing a reversible thermosensitive material A into microparticles/microgels and mixing them as a discrete phase with a continuous phase material B solution to obtain a multiphase printing ink; loading the multiphase printing ink into an extrusion-based printer, controlling the temperature to thermally soften the discrete phase into a sol state, and simultaneously performing extrusion printing to extrude the ink from the hopper, obtaining a discrete-continuous phase composite oriented fiber structure, and implementing 3D printed structure formation based on a pre-designed three-dimensional model; after printing is completed, performing optional and independent post-treatment cross-linking of the continuous phase and the discrete phase in the printed structure to obtain different three-dimensional structures with oriented characteristics.

### CROSS REFERENCE TO THE RELATED APPLICATIONS

This application is the national phase entry of International Application No. PCT/CN 2023/133526, filed on Nov. 23, 2023, the entire contents of which are incorporated herein by reference.

### TECHNICAL FIELD

This disclosure relates to the fields of advanced manufacturing and biomedicine, specifically a 3D printing method for in situ forming oriented fiber structures, and more particularly focusing on a 3D printing method capable of generating oriented subvoxel microstructures.

### BACKGROUND

Oriented anisotropic structures are widely found in nature, endowing structures with unique properties and functions. For instance, plants contain numerous vertically oriented channels that transport ions, water, and other components to upper tissues, facilitating nutrient delivery to meet the demands of photosynthesis [Zhu M, Song J, Li T, Gong A, Wang Y, Dai J, Yao Y, Luo W, Henderson D, and Hu L 2016 Highly Anisotropic, Highly Transparent Wood Composites. Advanced Materials 28 5181-7]. Similarly, animals possess tissues with oriented anisotropic structures, such as muscles, cartilage, tendons, nerves, blood vessels, and certain segments of the intestines [Khuu N, Kheiri S, and Kumacheva E 2021 Structurally anisotropic hydrogels for tissue engineering. Trends in Chemistry 3 1002-26; Xing J, Liu N, Xu N, Chen W, and Xing D 2022 Engineering Complex Anisotropic Scaffolds beyond Simply Uniaxial Alignment for Tissue Engineering. Advanced Functional Materials 32 2110676]. Compared to non-oriented structures, oriented structures not only play a crucial role in biological systems but also exhibit anisotropic characteristics in mechanical, electrical, thermal, and optical properties, offering unique advantages widely utilized in engineering applications. For example, the mechanical reinforcement properties provided by oriented structures are employed in the manufacturing of load-bearing steel bars [Chen J, Liu X, Tian Y, Zhu W, Yan C, Shi Y, Kong L B, Qi H J, and Zhou K 2022 3D-Printed Anisotropic Polymer Materials for Functional Applications. Advanced Materials 34 2102877], oriented porous structures are used in the development of supercapacitors [Wang F, Chen L, He S, Zhang Q, Liu K, Han X, Duan G, and Jiang S 2022 Design of wood-derived anisotropic structural carbon electrode for high-performance supercapacitor. Wood Science and Technology 56 1191-203], and oriented structures are also applied in the design and fabrication of sensors for equipment [Wang Z, Luan C, Liao G, Yao X, and Fu J 2019 Mechanical and self-monitoring behaviors of 3D printing smart continuous carbon fiber-thermoplastic lattice truss sandwich structure. Composites Part B: Engineering 176 107215] or wearable devices for the human body [Chen C, Wang Y, Wu Q, Wan Z, Li D, and Jin Y 2020 Highly strong and flexible composite hydrogel reinforced by aligned wood cellulose skeleton via alkali treatment for muscle-like sensors. Chemical Engineering Journal 400 125876]. Therefore, the construction of oriented structures holds significant importance.

3D printing, based on the concept of freeform fabrication, enables the creation of personalized and complex three-dimensional structures. Based on the characteristics of molding techniques, current 3D printing technologies can be broadly classified into powder bed fusion, vat photopolymerization, material jetting, material extrusion, and sheet lamination [Ligon S C, Liska R, Stampfl J, Gurr M, and Muelhaupt R 2017 Polymers for 3D Printing and Customized Additive Manufacturing. Chemical Reviews 117 10212-90]. Among these, material extrusion includes fused deposition modeling or solution extrusion, which can print a wide range of materials, including polylactic acid, acrylonitrile butadiene styrene, polyurethane, polyether ether ketone, etc. [Daminabo S C, Goel S, Grammatikos S A, Nezhad H Y, and Thakur V K 2020Fused deposition modeling-based additive manufacturing (3D printing): techniques for polymer material systems. Materials Today Chemistry 16 100248]. Solution extrusion supports the printing of materials with high water content, such as hydrogels and living cells [Zhang Y S, Haghiashtiani G, Hubscher T, Kelly D J, Lee J M, Lutolf M, McAlpine M C, Yeong W Y, Zenobi-Wong M, and Malda J 2021 3D extrusion bioprinting. Nature Reviews Methods Primers 1 75]. Therefore, 3D printing has been applied to the fabrication of oriented structures with various materials.

Traditional material extrusion printing methods typically produce material filaments with unit dimensions generally above 100 micrometers, limiting the minimum feature size of the oriented structures that can be constructed [Ligon S C, Liska R, Stampfl J, Gurr M, and Muelhaupt R 2017 Polymers for 3D Printing and Customized Additive Manufacturing. Chemical Reviews 117 10212-90]. Traditional material extrusion printing processes can reduce the size of material filaments under the influence of an electric field: electrospinning technology can generate micrometer-scale filaments, achieving a certain degree of orientation, but with limited control over orientation and difficulty in achieving precise deposition of filaments, making it challenging to form thick structures [Sun B, Long Y Z, Zhang H D, Li M M, Duvail J L, Jiang X Y, and Yin H L 2014 Advances in three-dimensional nanofibrous macrostructures via electrospinning. Progress in Polymer Science 39 862-90]; near-field electrohydrodynamic printing technology can control the arrangement of micrometer-scale filaments, achieving good orientation, but is also limited by manufacturing speed and difficulty in forming thick structures [Kade J C and Dalton P D 2021 Polymers for Melt Electrowriting. Advanced Healthcare Materials 10 2001232].

Existing technologies achieve fiber orientation by pre-loading fine fibers within the extruded material and utilizing extrusion shearing forces. However, short fibers are generally employed, and the alignment of fiber ends is uncontrollable, making it difficult to achieve a distribution of oriented continuous fibers with a large aspect ratio. For instance, after fabricating a fiber membrane from norbornene-modified hyaluronic acid using electrospinning technology, it can be chopped to obtain short fibers with lengths less than 20 micrometers. These short fibers, when mixed with bioink and printed via extrusion, can rearrange along the extrusion direction to form an oriented arrangement. However, the continuity of these short fibers remains an issue to be addressed [Prendergast M E, Davidson M D, and Burdick J A 2021 A biofabrication method to align cells within bioprinted photocrosslinkable and cell-degradable hydrogel constructs via embedded fibers. Biofabrication 13 044108]. Other technologies extrude materials from an aqueous two-phase system that are immiscible with each other. Under the action of extrusion shearing, the two materials form stratified and oriented flows, resulting in structures with a certain degree of orientation. However, the uniformity of the internal oriented porous structure sizes is poor. For example, using a mixed material system of gelatin methacryloyl and polyethylene oxide, stratified oriented fibers can be obtained through coaxial extrusion and photocuring cross-linking. However, there is a noticeable phenomenon of interlayer connectivity within the oriented fibers [Shao L, Hou R, Zhu Y, and Yao Y 2021 Pre-shear bioprinting of highly oriented porous hydrogel microfibers to construct anisotropic tissues. Biomaterials Science 9 6763-71]. Additionally, some utilize Kenics static mixers to achieve stratification of two different materials, resulting in fiber structures with an oriented effect. Specifically, two materials are simultaneously injected into the mixer, and during the flow mixing process, stratification occurs. Cross-linking and fixation of the stratified fibers can yield oriented fibers. However, the internal oriented structure is lamellar, making it difficult to obtain filamentous substructures [Bolívar-Monsalve E J, Ceballos-González C F, Borrayo-Montaño K I, Quevedo-Moreno D A, Yee-de León J F, Khademhosseini A, Weiss P S, Alvarez M M, and Trujillo-de Santiago G 2021 Continuous chaotic bioprinting of skeletal muscle-like constructs. Bioprinting 21 e00125; Bolívar-Monsalve E J, Ceballos-González C F, Chávez-Madero C, de la Cruz-Rivas B G, Velásquez Marín S, Mora-Godínez S, Reyes-Cortés L M, Khademhosseini A, Weiss P S, Samandari M, Tamayol A, Alvarez M M, and Trujillo-de Santiago G 2022 One-Step Bioprinting of Multi-Channel Hydrogel Filaments Using Chaotic Advection: Fabrication of Pre-Vascularized Muscle-Like Tissues. Advanced Healthcare Materials 11 2200448]. In summary, constructing continuous micron-scale oriented structures within 3D-printed models remains challenging.

### SUMMARY

In this disclosure, a reversible thermosensitive material A is fabricated into microparticles/microgels, which are then mixed as a discrete phase with a continuous phase material B solution to form a multiphase printing ink. The multiphase printing ink is loaded into an extrusion-based printer. By controlling the temperature, the discrete phase microparticles/microgels are thermally softened into a sol state, and simultaneous extrusion printing is carried out to extrude the ink from the hopper. During the extrusion process, the discrete phase undergoes stretch deformation to form fine fibers, resulting in an oriented fiberous structure composed of a discrete-continuous phase composite. Based on a pre-designed three-dimensional (3D) model, 3D printing structural shaping is performed. After printing is completed, optional and independent post-treatment cross-linking is conducted on the continuous phase and the discrete phase within the printed structure, yielding different 3D structures with oriented characteristics.

The challenge lies in the fact that existing 3D printing technologies struggle to generate continuous micron-scale oriented structures within their printed constructs.

The objective of this disclosure is to provide a novel 3D printing method that can couple the phase transition and deformation of multiphase inks to form oriented fiber microstructures in situ, thereby addressing the challenge of generating continuous micron-scale oriented structures within 3D-printed constructs.

The 3D printing method provided by this disclosure for forming oriented fiber structures in situ includes the following steps:
1) fabricating reversible thermosensitive material A into microparticles/microgels, and mixing them as a discrete phase with a continuous phase material B solution to obtain multiphase printing ink;
2) loading the multiphase printing ink into an extrusion-based printer, controlling the temperature to thermally soften the discrete phase microparticles/microgels into a sol state, and simultaneously performing extrusion printing to extrude the ink from the hopper, wherein during the extrusion process, the discrete phase undergoes stretch deformation to form fine fibers, resulting in an oriented fiberous structure composed of a discrete-continuous phase composite. Based on a pre-designed 3D model, implement 3D printing structural shaping;
3) after printing is completed, performing optional and independent post-treatment cross-linking on the continuous phase and the discrete phase within the printed structure to obtain different 3D structures with oriented characteristics.

In step 1) of the above method, the reversible thermosensitive material A can be at least one of the following 1)-3): 1) gelatin or modified gelatin or their mixtures; 2) a mixture of gelatin and a blending phase; a mixture of modified gelatin and a blending phase; a mixture of gelatin and modified gelatin and a blending phase, wherein the blending phase can be one or more of hyaluronic acid, silk fibroin, heparin, polyethylene glycol, dextran, chondroitin sulfate, sodium alginate, chitosan, and fibrinogen; 3) poly(N-isopropylacrylamide) and its modified products.

The reversible thermosensitive material A may also contain hydrogel materials that can form stable structures through chemical or physical cross-linking, which can serve as the main component or additive of the discrete phase microparticles/microgels.

Further, the hydrogel material that can form a stable structure through chemical or physical cross-linking may specifically be gelatin methacryloyl.

The method for fabricating the reversible thermosensitive material A into microparticles/microgels is not limited and can be selected from any of the following: droplet microfluidics, oil-in-water emulsion method, complex coacervation method, electrospraying method, mechanical fragmentation method, etc.

Firstly, a solution of the reversible thermosensitive material A is prepared, and then it is fabricated into microparticles/microgels using any of the aforementioned methods.

In the solution of the reversible thermosensitive material A, the mass concentration of the reversible thermosensitive material A can range from 2% to 20%, preferably 10%.

A 0.9% sodium chloride solution, phosphate-buffered saline, or ultrapure water can be used as the solvent to prepare the solution of the reversible thermosensitive material A.

The morphology of the microparticles/microgels can be spherical (with diameters ranging from 10 to 5000 micrometers), polyhedral, or other irregular three-dimensional shapes (with volumes ranging from 1000 cubic micrometers to 125 cubic millimeters); specifically, they can be spherical with diameters ranging from 100 to 250 micrometers.

The continuous phase material B maintains extrudability within the operating temperature range and exhibits poor mutual solubility with the reversible thermosensitive material A.

Specifically, the continuous phase material B can be selected from the following: 1) Pluronic or modified Pluronic or their mixtures; 2) a mixture of Pluronic and a blending phase; a mixture of modified and a blending phase; a mixture of Pluronic, modified Pluronic and a blending phase, wherein the blending phase can be one or more of hyaluronic acid, silk fibroin, heparin, polyethylene glycol, dextran, chondroitin sulfate, sodium alginate, chitosan, fibrinogen, etc.; 3) Polyethylene glycol or modified polyethylene glycol or their mixtures.

The continuous phase material B may also contain photosensitive cross-linking components to facilitate subsequent post-treatment cross-linking of the continuous phase.

Preferably, the continuous phase material B contains photosensitive modified Pluronic.

When the continuous phase material B is Pluronic or modified Pluronic or their mixtures, the mass concentration of Pluronic or modified Pluronic or their mixtures in the solution of the continuous phase material B ranges from 10% to 50%, preferably 30%.

When the continuous phase material B is polyethylene glycol or modified polyethylene glycol or their mixtures, the mass concentration of polyethylene glycol or modified polyethylene glycol or their mixtures in the solution of the continuous phase material B ranges from 5% to 50%.

The solution of the continuous phase material B also contains a photoinitiator, with a mass concentration of the photoinitiator ranging from 0.05% to 0.5%.

The photoinitiator can specifically be lithium phenyl (2,4,6-trimethylbenzoyl) phosphinate.

The solution of the continuous phase material B can use 0.9% sodium chloride solution, phosphate-buffered saline, or ultrapure water as the solvent.

The mass-to-volume mixing ratio range of the discrete phase to the solution of the continuous phase material B can be from 1 g:2 ml to 1 g:128 ml, preferably 1 g:8 ml.

Temperature control involves maintaining the ink hopper or nozzle above the critical temperature, which refers to the temperature at which the discrete phase microparticles/microgels are thermally softened into a sol state. The critical temperature is at or above the sol temperature/melting temperature of material A.

For reversible thermosensitive materials with different compositions or concentrations, the critical temperature varies.

When the reversible thermosensitive material A is primarily gelatin or modified gelatin, the critical temperature is 32 °C or higher.

When the reversible thermosensitive material A is poly (N-isopropylacrylamide) and its modified products, the critical temperature is 32 °C or higher.

The nozzle diameter ranges from 0.2 to 2 mm, preferably 0.26 mm, and the printing speed is controlled between 5 and 20 mm/s, preferably 10 mm/s.

The operation in step 3) involves post-crosslinking treatment of the discrete phase in the printed structure, cross-linking the hydrogel material that can form a stable structure through chemical or physical cross-linking contained in the microparticles/microgels of the reversible thermosensitive material A, to obtain a hydrogel structure with oriented fine fibers inside.

Alternatively, post-crosslinking treatment of the continuous phase in the printed structure is performed, and the hydrogel structure after post-crosslinking treatment of the continuous phase is soaked above the dissolution temperature of the reversible thermosensitive material A, allowing the reversible thermosensitive material A to dissolve and form oriented channels inside the hydrogel structure, resulting in an oriented channel hydrogel structure.

Alternatively, optional cross-linking of the continuous phase in the printed structure and the hydrogel material contained in the microparticles/microgels of the reversible thermosensitive material A is performed (specific operation: selective irradiation of the printed structure through a mask to remove the uncured continuous phase), resulting in customized oriented fiber scaffold.

Among them, post-crosslinking treatment of the continuous phase in the printed structure is achieved through light irradiation, with the light source wavelength ranging from 200 to 410 nm, specifically 405 nm, and the irradiation time ranging from 10 to 300 seconds, preferably 180 seconds.

The three-dimensional structures with oriented characteristics (hydrogel structures with oriented fine fibers inside, oriented channel-embedded hydrogel structures, customized oriented fiber scaffolds) produced by the aforementioned method also fall within the scope of protection of this disclosure.

The preparation method of the present disclosure offers the following advantages over the existing technologies.
1. The multiphase ink in the present disclosure can obtain complex 3D structures through a printing process that couples phase transition and deformation, enabling the manufacturing of three-dimensional solid objects with personalized oriented fine fiber or fine fiber bundle structures.
2. It allows for the fabrication of three-dimensional oriented micron-scale hydrogel structures, oriented channel structures, separated fibers, or fiber bundles through selective cross-linking.
3. It can be combined with 3D bioprinting to achieve the preparation of cell-laden three-dimensional structures with oriented characteristics under cell-loaded conditions.

### BRIEF DESCRIPTION OF THE DRAWINGS

FIG. 1 is a flowchart of the 3D printing process for in-situ formation of oriented fiber structures in the present disclosure.

FIG. 2 is a schematic diagram of the technical solution for the 3D printing method for in-situ formation of oriented fiber structures in the present disclosure.

FIG. 3 is a schematic diagram illustrating the fabrication of oriented fiber/channel hydrogel structures in Example 1 of the present disclosure.

FIG. 4 shows the influence of printing temperature on the extrusion printing process of the multiphase ink in Example 1 of the present disclosure.

FIG. 5 is a 3D hydrogel model with oriented fiber substructures prepared in Example 1 of the present disclosure.

FIG. 6 is a diameter distribution diagram of microfibers within the oriented structures prepared in Example 1 of the present disclosure.

FIG. 7 presents the cell viability statistics in the oriented cell-laden hydrogel structures prepared in Example 2 of the present disclosure.

FIG. 8 is a staining diagram of elongated myotubes with a high aspect ratio in the oriented cell-laden hydrogel structures prepared in Example 2 of the present disclosure.

FIG. 9 is a technical schematic diagram illustrating the preparation of separated fiber bundle in Example 3 of the present disclosure.

FIG. 10 is a technical schematic diagram illustrating the preparation of oriented fiber scaffolds in Example 3 of the present disclosure.

FIG. 11 shows the oriented fiber scaffolds after removal of the continuous phase in Example 3 of the present disclosure.

FIG. 12 illustrates the induction of cell elongation by the oriented fiber scaffolds in Example 3 of the present disclosure.

FIG. 13 is a schematic diagram of the oriented channel scaffolds prepared in Example 4 of the present disclosure.

FIG. 14 depicts the oriented hydrogel microfiber structures based on modular discrete phase multiphase inks in the present disclosure.

FIG. 15 shows the fabrication of oriented heterogeneous structures based on modular discrete phase multiphase inks in the present disclosure.

FIG. 16 illustrates the fabrication of oriented hydrogel microfiber structures using discrete phases based on physically cross-linked characteristic materials in the present disclosure.

FIG. 17 illustrates the fabrication of oriented hydrogel microfiber structures using continuous phases based on physically cross-linked characteristic materials in the present disclosure.

### DETAILED DESCRIPTION OF THE EMBODIMENTS

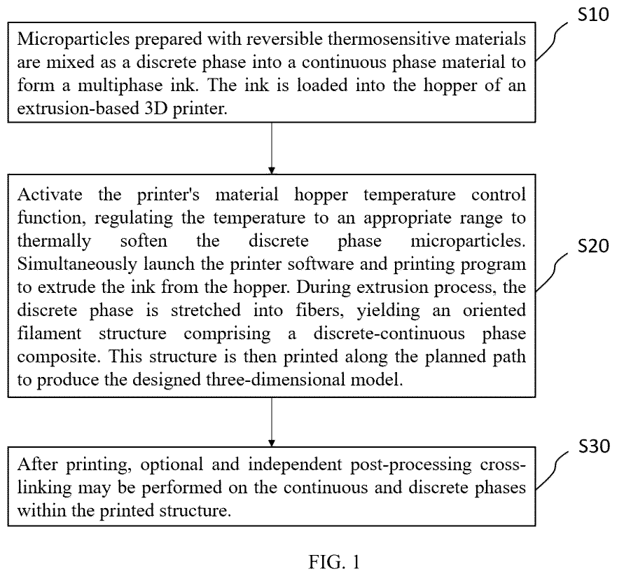

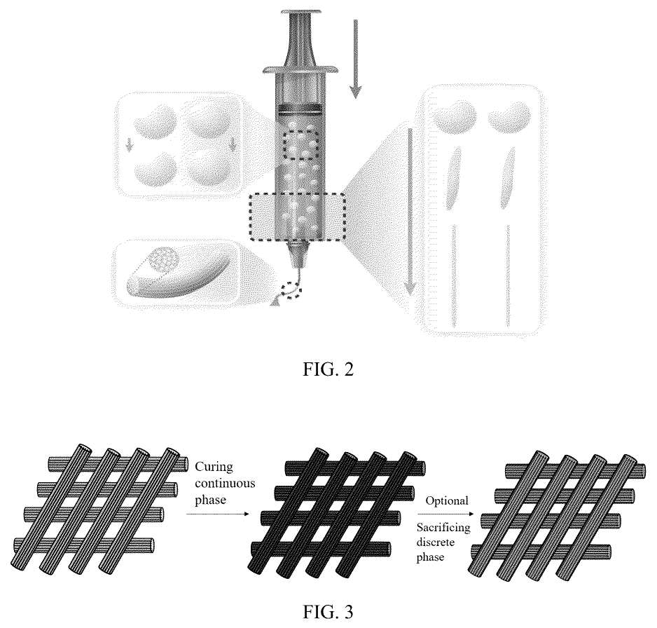

The present disclosure employs a multiphase ink system. During the extrusion printing process, it utilizes the in-situ shear and extensional effect to stretch the discrete phase particles within the ink into fibers with a high aspect ratio, constructing fine oriented fibers within the extruded filament units and achieving a hierarchical orientation outcome (FIG. 1 and FIG. 2).

Basic operational procedure: Prepare microparticles A as the discrete phase and mix them with a continuous phase solution B to form a multiphase printing ink (S10). Load this ink into an extrusion printer, adjust the solid A to a fluid state (solid-liquid transition) through temperature control, and simultaneously perform extrusion printing. Phase A undergoes deformation during flow, forming fine fibers. The finally extruded filament contains highly oriented fine fibers of the discrete phase material A within the continuous phase material B. Subsequently, under computer control, 3D printed structure formation is carried out based on a pre-designed three-dimensional model (S20). After printing, optional and independent post-treatment cross-linking of the continuous and discrete phases in the printed structure can be performed (S30) (FIG. 1).

The methacrylated gelatin in the following examples is prepared by the following method:
1. adding 1 g of solid gelatin to 10 ml of ultrapure water and stir at 50 °C until completely dissolved to obtain a gelatin solution;
2. while maintaining stirring at 50 °C, dropwise adding 0.6 ml of methacrylic anhydride solution to the gelatin solution from step 1 and continue stirring for 3 hours;
3. after the reaction is complete, transferring the solution from step 2 to a 50-ml centrifuge tube to remove unreacted methacrylic acid, centrifuging at 2000 rpm for 3 minutes at room temperature, transferring the supernatant solution to a glass beaker, and adding 30 ml of ultrapure water to dilute the supernatant;
4. transferring the solution from step 3 to a dialysis bag with a molecular weight cutoff of 8000-14000 Da and dialyze at 40 °C for 7 days;
5. adjusting the pH of the solution from step 4 to 7.4 using a 1 M NaOH solution;
6. after freeze-drying, obtaining the methacrylated gelatin material.

The fluorescent gelatin is prepared by the following method:
1. adjusting the pH of a phosphate-buffered saline solution to 8.1 by adding a 1 M NaOH solution and set aside;
2. dissolving 1 g of gelatin in 5 ml of the alkaline phosphate-buffered saline solution from step 1 and stirring at 50 °C to prepare a gelatin solution;
3. adding 30 mg of fluorescein material (fluorescein isothiocyanate) to 5 ml of the alkaline phosphate-buffered saline solution from step 1 and stirring at 50 °C until dissolved;
4. dropwise adding the fluorescein solution from step 3 to the gelatin solution from step 2 and stirring at 50 °C in a light-shielded environment for 3 hours;
5. quenching the reaction with 40 ml of PBS, transferring the reaction liquid to a dialysis bag, and dialyzing at 40 °C for 7 days.
6. after freeze-drying, obtaining the solid fluorescent gelatin material.

### Example 1: Fabrication of Oriented Microfiber/channel-embedded Hydrogel Structures

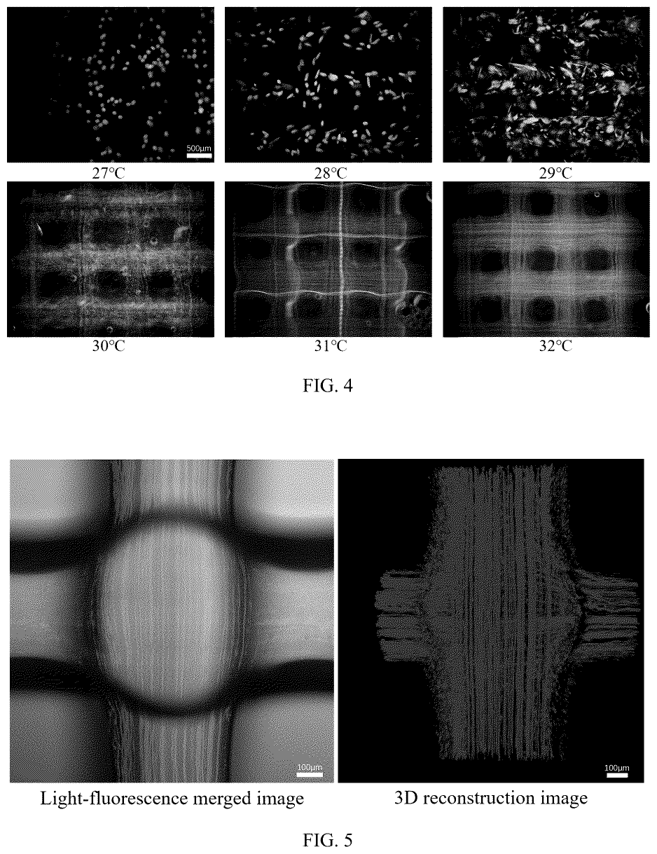

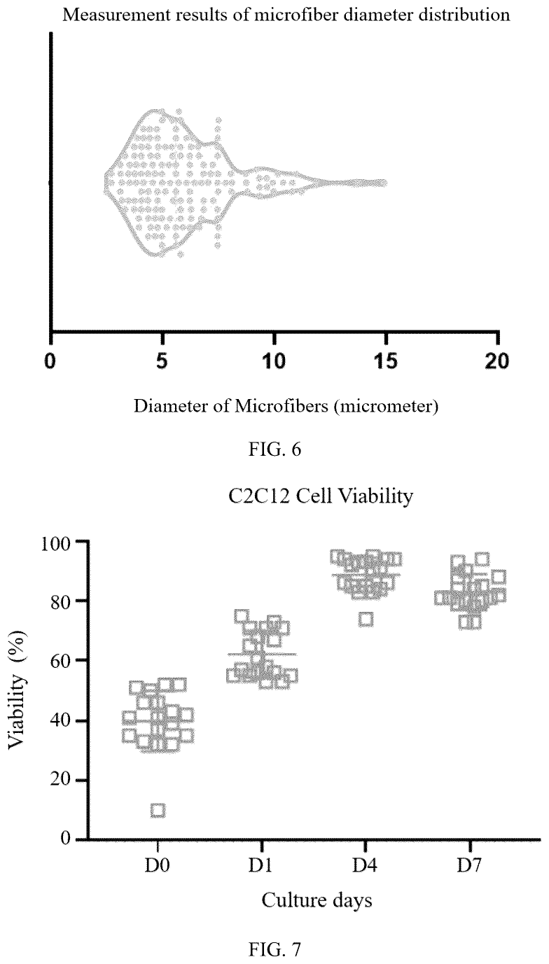

The schematic diagram is shown in FIG. 3.
1. Preparation of fluorescent gelatin thermosensitive microgels: the method for preparing gelatin microgels is not limited and can include droplet microfluidics, oil-in-water emulsion, complex coacervation, electrospraying, mechanical fragmentation, etc. In this example, droplet microfluidics is used for the preparation of thermosensitive gelatin microgels. A 10% (w/w) fluorescent gelatin solution is prepared using a 0.9% sodium chloride solution as the solvent, and gelatin solids are dissolved at 60 °C. By adjusting the flow rates of the self-made droplet microfluidic device, with an oil flow rate of 8 mL/h and a gelatin solution flow rate of 1 mL/h, microgels with a stable diameter of approximately 200 micrometers are obtained. The microgels are concentrated by centrifugation and further washed with a cleaning solution (0.9% sodium chloride solution) to remove oil-phase components on the surface of the microgels. Through repeated concentration-washing steps, the oil-phase content is reduced to a level that did not affect subsequent steps.
2. Preparation of the photosensitive modified Pluronic continuous phase solution: A 30% (w/w) photosensitive modified Pluronic solution is prepared using a 0.9% sodium chloride solution as the solvent, containing 0.15% of the photoinitiator lithium phenyl(2,4,6-trimethylbenzoyl) phosphinate. The photosensitive modified Pluronic solids are dissolved at 0-4 °C to obtain the corresponding concentration of the photosensitive modified Pluronic continuous phase solution.
3. Preparation and printing of the multiphase ink system: the thermosensitive gelatin microgels are mixed with the photosensitive modified Pluronic solution at a mass-to-volume ratio of 1 g:8 mL. After thorough mixing, the mixture is loaded into the nozzle of an extrusion printer for printing. The temperatures of the nozzle and the receiving substrate are controlled at 32 °C, respectively. The nozzle diameter is 0.26 mm, and the printing speed is controlled at 10 mm/s. After printing, the hydrogel structure is irradiated with a 405 nm wavelength light source for 180 seconds to cross-link the photosensitive modified Pluronic.
4. Subsequently, the scaffold is gently washed three times with a 4 °C PBS solution to remove excess cross-linker. The printed hydrogel structure is recorded under a confocal microscope, and the shape of the internal thermosensitive gelatin microgels is related to the printing temperature (FIG. 4). At or above the appropriate temperature (referred to as the critical temperature), the thermosensitive gelatin microspheres could stably transform into oriented fine fibers parallel to the extrusion direction. With the movement of the 3D printer, hydrogel filaments with oriented fiber structures could be stacked and deposited to form a three-dimensional structure (FIG. 5), with the vast majority of the internal oriented fine fibers having a diameter of less than 10 micrometers (FIG. 6).
5. In this example, the material composition of the thermosensitive microgels could be controlled, allowing for the optional sacrifice or retention of the oriented structures. In scenarios where sacrifice is chosen, gelatin is used as the thermosensitive microgels. The oriented hydrogel structure obtained by printing above the critical temperature is soaked in 37 °C PBS, and the oriented gelatin fibers dissolved and flowed out, forming oriented channels within the hydrogel structure. The dissolution rate of gelatin is positively correlated with the volume and temperature of the soaking liquid. In scenarios where retention is chosen, hydrogel materials that could form stable structures through chemical or physical cross-linking were used as the main component or additive of the thermosensitive microgels. After printing above the critical temperature and subsequent cross-linking, a hydrogel structure with internal oriented fine fibers could be obtained. In this example, methacrylated gelatin is used as the material for the thermosensitive microgels, and the obtained oriented fine fibers remained well-preserved after 10 days at 37 °C.

### Example 2: Fabrication of Oriented Cell-Laden Hydrogel Structures

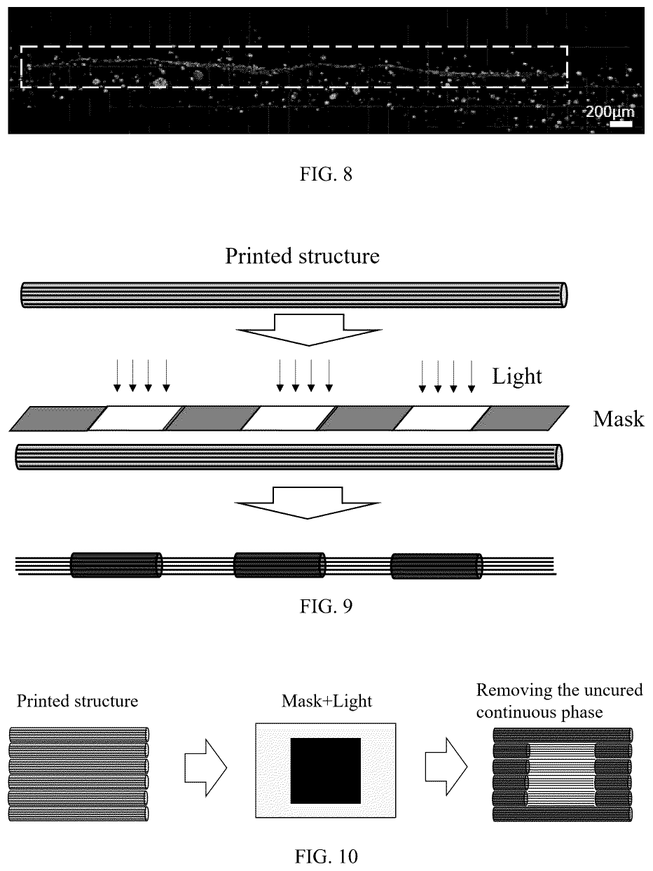

1. As in Steps 1 and 2 of Example 1, a 10% (w/w) gelatin solution and thermosensitive gelatin microgels with a diameter of approximately 200 micrometers are prepared; a solution containing 5% (w/w) photosensitive modified Pluronic and 25% (w/w) regular Pluronic (non-photosensitive) with 0.15% of the photoinitiator [lithium phenyl (2,4,6-trimethylbenzoyl) phosphinate] is also prepared.
2. The mouse myoblast cell line (abbreviated as “C2C12”) is cultured in DMEM high-glucose medium supplemented with 10% fetal bovine serum. The medium is changed every 2 days. Once the cells reach the logarithmic growth phase, they are digested with 0.25% trypsin, centrifuged, and collected for further use.
3. Preparation of the multiphase ink system: the thermosensitive gelatin microgels and the Pluronic solution are added to a sterile centrifuge tube at a mass-to-volume ratio of 1 g:8 mL, they are mixed thoroughly and set aside for later use.
4. Preparation of the cell bioink: the C2C12 cells are resuspended using the multiphase ink system obtained in Step 3 to achieve a bioink concentration of 5×10⁶ cells/mL. After thoroughly mixing, the bioink is loaded into a pre-sterilized extrusion printer.
5. Printing of the cell-laden hydrogel structures: a nozzle diameter of 0.26 mm is selected and the printing speed is controlled at 5 mm/s, with a printing temperature of 32 °C. After printing, the hydrogel structures are irradiated with a 405 nm wavelength light source for a duration of 30 seconds to cross-link the photosensitive modified Pluronic.
6. Subsequently, the cell-laden hydrogel structures are gently soaked and washed three times with 4 °C PBS solution for 10 minutes per time to remove excess cross-linker and uncross-linked Pluronic. The printed cell structures are then transferred to a culture dish/well plate, and DMEM high-glucose medium supplemented with 10% fetal bovine serum is added for culture. Every 48 hours, the medium soaking the hydrogel scaffold is replaced with fresh medium.
7. The cell status within the structures is regularly observed and the growth state of the C2C12 cells is evaluated. The cell viability remains good within seven days (FIG. 7). After seven days of culture, the cells fuse to form myotubes with a large aspect ratio (FIG. 8).

### Example 3: Fabrication of Oriented Hydrogel Microfiber Structures with Partial Cross-linking of the Continuous Phase

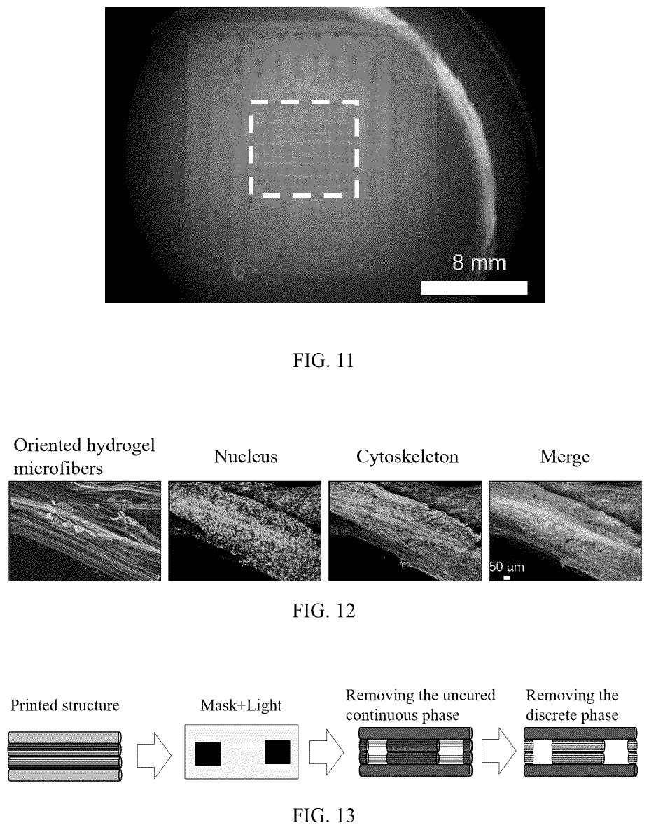

1. As in Steps 1 and 2 of Example 1, a 10% (w/w) methacrylated gelatin solution and methacrylated gelatin thermosensitive microgels with a diameter of approximately 200 micrometers are prepared; a solution containing 5% (w/w) photosensitive modified Pluronic and 25% (w/w) regular Pluronic (non-photosensitive) with 0.15% of the photoinitiator [lithium phenyl (2,4,6-trimethylbenzoyl) phosphinate] is prepared.
2. Preparation and printing of the multiphase ink system: the methacrylated gelatin thermosensitive microgels are mixed with the Pluronic solution at a mass-to-volume ratio of 1 g:8 mL. After thoroughly mixing, the mixture is loaded into the nozzle of an extrusion printer for printing. The temperatures of the nozzle and the receiving substrate are both controlled at 32 °C. A nozzle diameter of 0.26 mm is selected, the printing speed is controlled at 10 mm/s, and the printing temperature is maintained at or above the critical temperature. After printing, the structure is selectively irradiated through a mask with a 405 nm wavelength light source for a duration of 20 seconds to optionally cross-link the photosensitive modified Pluronic, resulting separated fiber bundle (FIG. 9) or oriented fiber scaffolds (FIG. 10).
3. The hydrogel structure is gently soaked and washed three times with a 4 °C PBS solution for 10 minutes each time to remove excess cross-linker and uncross-linked Pluronic.
4. After washing, the hydrogel structure is resoaked in 4 °C PBS containing 0.15% photoinitiator. The hydrogel structure is irradiated with a 405 nm wavelength light source for a second time to achieve secondary cross-linking of the methacrylated gelatin fiber structure. The final oriented fiber scaffold after removal of the uncross-linked continuous phase is shown in FIG. 11.
5. The oriented fiber scaffold structure in this example can induce cell elongation. As described in Step 2 of Example 2, a 5×10⁶ cells/mL C2C12 cell suspension is prepared. The cell suspension is added onto the oriented fiber scaffold structure prepared in this example for culture. Every 48 hours, the medium soaking the scaffold is replaced with fresh medium.
6. The cell status within the structure is regularly observed and the growth state of the C2C12 cells is evaluated. The cells are able to adhere and elongate on the oriented fiber scaffold structure (FIG. 12).

### Example 4: Fabrication of Oriented Hydrogel Microfiber Structures with Partial Cross-linking of Both Continuous and Discrete Phases

1. As described in Steps 1 and 2 of Example 1, a 10% (w/w) gelatin solution and gelatin thermosensitive microgels with a diameter of approximately 200 micrometers are prepared; additionally, a solution containing 5% (w/w) photosensitive modified Pluronic, 25% (w/w) regular Pluronic (non-photosensitive), and 0.15% of the photoinitiator [lithium phenyl (2,4,6-trimethylbenzoyl) phosphinate] is prepared.
2. Preparation and printing of the multiphase ink system: the gelatin thermosensitive microgels are mixed with the Pluronic solution at a mass-to-volume ratio of 1 g:8 mL. After thorough mixing, the mixture is loaded into the nozzle of an extrusion printer for printing. The temperatures of both the nozzle and the receiving substrate are controlled at 32 °C. A nozzle diameter of 0.26 mm is selected, the printing speed is controlled at 10 mm/s, and the printing temperature is maintained at or above the critical temperature. After printing, the hydrogel structure is selectively irradiated through a mask using a 405 nm wavelength light source (FIG. 13) for a duration of 20 seconds to optionally cross-link the photosensitive modified Pluronic.
3. The hydrogel structure is gently soaked and washed three times with a 4 °C PBS solution for 10 minutes each time to remove excess cross-linker and uncross-linked Pluronic. Then, the hydrogel structure is immersed at a temperature above 37 °C. The oriented gelatin fibers will dissolve and flow out, creating oriented channels within the hydrogel structure and resulting in an oriented fiber-pore scaffold.

### Example 5: Fabrication of a Composite Oriented Hydrogel Microfiber-Oriented and Channel-Embedded Structure

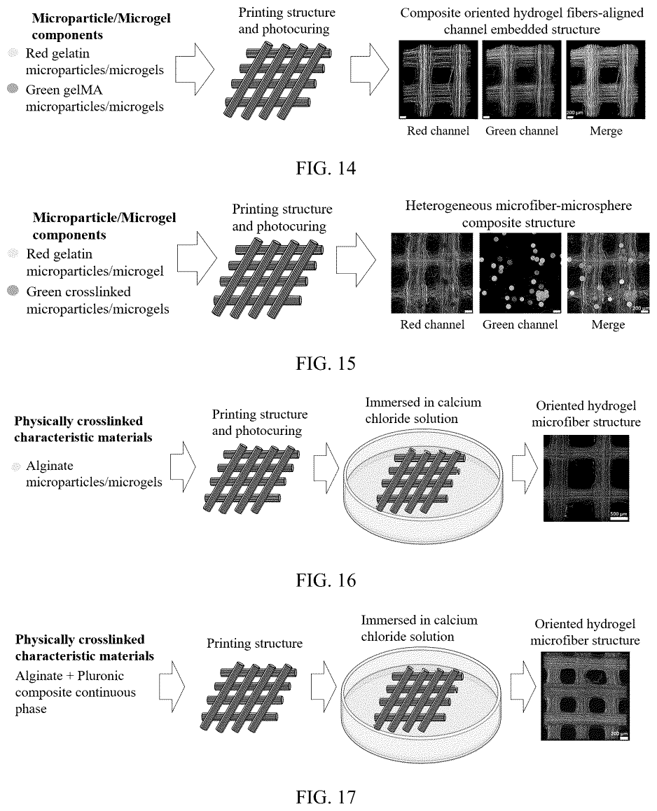

1. As described in Steps 1 and 2 of Example 1, a 10% (w/w) red fluorescent gelatin solution and a 10% (w/w) green fluorescent methacrylated gelatin solution are prepared separately. Additionally, red gelatin thermosensitive microgels and green methacrylated gelatin thermosensitive microgels, each with a diameter of approximately 200 micrometers, are prepared. Furthermore, a solution containing 5% (w/w) photosensitive modified Pluronic, 25% (w/w) regular Pluronic (non-photosensitive), and 0.15% of the photoinitiator [lithium phenyl (2,4,6-trimethylbenzoyl) phosphinate] is prepared.
2. Preparation and printing of the multiphase ink system; the red gelatin thermosensitive microgels and green methacrylated gelatin thermosensitive microgels are mixed in a mass ratio determined by the application requirements; in this example, a mass ratio of 1 g:1 g is used for verification. After obtaining the gelatin thermosensitive microgels of two colors, they are mixed with the Pluronic solution at a mass-to-volume ratio of 1 g:8 mL. After thoroughly mixing, the mixture is loaded into an extrusion printer for printing. The temperatures of both the nozzle and the receiving substrate are controlled at 32 °C. A nozzle diameter of 0.26 mm is selected, the printing speed is controlled at 10 mm/s, and the printing temperature is maintained at or above the critical temperature. After printing, the hydrogel structure is irradiated with a 405 nm wavelength light source for a duration of 180 seconds to cross-link the photosensitive modified Pluronic and the oriented methacrylated gelatin microfibers (FIG. 14).
3. The hydrogel structure is gently soaked and washed three times with a 4 °C PBS solution for 10 minutes each time to remove excess cross-linker and uncross-linked Pluronic. Then, the hydrogel structure is immersed at a temperature above 37 °C. The red gelatin oriented microfibers will dissolve and flow out, creating oriented channels within the hydrogel structure, while the green methacrylated gelatin oriented microfibers will remain intact, forming an oriented fiber structure within the hydrogel. Finally, a composite scaffold with both oriented channels and oriented fibers is obtained.
4. In this example, modular discrete-phase ink compositions can be achieved by combining microparticles/microgels prepared from different materials. Through the printing process described in Step 2, a composite oriented hydrogel structure composed of multi-material hydrogel fibers can be realized, where the cross-linking method of the hydrogel fibers is determined by the materials of the discrete-phase microparticles/microgels.

### Example 6: Fabrication of a Composite Hydrogel Heterogeneous-Oriented Structure

1. As described in Step 1 of Example 1, 10% (w/w) red fluorescent gelatin solution and 10% (w/w) green fluorescent methacrylated gelatin solution are prepared separately. Additionally, red gelatin thermosensitive microgels and green methacrylated gelatin thermosensitive microgels, each with a diameter of approximately 200 micrometers, are prepared. The green methacrylated gelatin thermosensitive microgels are pre-soaked in a solution containing 0.15% of the photoinitiator [lithium phenyl (2,4,6-trimethylbenzoyl) phosphinate], and then irradiated with a 405 nm wavelength light source for 180 seconds to cross-link the green methacrylated gelatin thermosensitive microgels.
2. As described in Step 2 of Example 1, a solution containing 5% (w/w) photosensitive modified Pluronic, 25% (w/w) regular Pluronic (non-photosensitive), and 0.15% of the photoinitiator [lithium phenyl (2,4,6-trimethylbenzoyl) phosphinate] is prepared.
3. Preparation and printing of the multiphase ink system: the red gelatin thermosensitive microgels and green methacrylated gelatin microgels are pre-mixed in a mass ratio determined by the application requirements; in this example, a mass ratio of 1 g: 1 g is used for verification. After the microgels of two colors are obtained, they are mixed with the Pluronic solution at a mass-to-volume ratio of 1 g:8 mL. After thoroughly mixing, the mixture is loaded into an extrusion printer for printing. The temperatures of both the nozzle and the receiving substrate are controlled at 32 °C. A nozzle diameter of 0.26 mm is selected, the printing speed is controlled at 10 mm/s, and the printing temperature is maintained at or above the critical temperature. After printing, the hydrogel structure is irradiated with a 405 nm wavelength light source for 180 seconds to cross-link the photosensitive modified Pluronic (FIG. 15).
4. The hydrogel structure is gently soaked and washed three times with a 4 °C PBS solution for 10 minutes each time to remove excess cross-linker and uncross-linked Pluronic. Then, the hydrogel structure is immersed at a temperature above 37 °C. The red gelatin oriented fibers will dissolve and flow out, creating oriented channels within the hydrogel structure, while the green methacrylated gelatin microspheres will remain intact, forming a microsphere structure within the hydrogel. Finally, a composite scaffold with both oriented channels and microspheres is obtained.
5. In this example, by combining microparticles/microgels with thermosensitive and non-thermosensitive properties as modular discrete-phase ink compositions, a composite hydrogel heterogeneous-oriented structure with multi-material compositions can be fabricated through the printing process described in Step 2. The internal structure of the resulting hydrogel is a composite of oriented structures and the shapes of the microparticles/microgels.

### Example 7: Fabrication of an Oriented Hydrogel Microfiber Structure with a Discrete Phase Based on Physically Cross-linked Characteristic Materials

1. As described in Steps 1 and 2 of Example 1, a mixed solution containing 10% (w/w) gelatin +0.5% (w/w) alginic acid, and gelatin-alginic acid thermosensitive microgels with a diameter of approximately 200 micrometers are prepared. Additionally, a solution containing 5% (w/w) photosensitive modified Pluronic, 25% (w/w) regular Pluronic (non-photosensitive), and 0.15% of the photoinitiator [lithium phenyl (2,4,6-trimethylbenzoyl) phosphinate] is prepared.
2. Preparation and printing of the multiphase ink system: the gelatin-alginic acid thermosensitive microgels are mixed with the Pluronic solution at a mass-to-volume ratio of 1 g:8 mL. After thoroughly mixing, the mixture is loaded into an extrusion printer for printing. The temperatures of both the nozzle and the receiving substrate are controlled at 32 °C. A nozzle diameter of 0.26 mm is selected, the printing speed is controlled at 10 mm/s, and the printing temperature is maintained at or above the critical temperature. After printing, the hydrogel structure is selectively irradiated through a mask using a 405 nm wavelength light source for 20 seconds to optionally cross-link the photosensitive modified Pluronic (FIG. 16).
3. The hydrogel structure is soaked in a deionized water solution containing 3% calcium chloride for 3 minutes to cross-link the oriented alginic acid hydrogel microfibers. Then, the hydrogel structure is gently soaked and washed three times with a 4 °C PBS solution for 10 minutes each time to remove excess cross-linker and uncross-linked Pluronic, thereby achieving the fabrication of an oriented hydrogel microfiber structure based on physically cross-linked characteristic materials.

### Example 8: Fabrication of an Oriented Hydrogel Microfiber/Channel Structure with a Continuous Phase Based on Physically Cross-linked Characteristic Materials

1. As described in Step 1 of Example 1, a 10% (w/w) gelatin solution and gelatin thermosensitive microgels with a diameter of approximately 200 micrometers are prepared.
2. A composite continuous phase solution including alginic acid and Pluronic is prepared by using a 0.9% sodium chloride solution as the solvent to dissolve alginic acid, thereby formulating a 1% (w/w) alginic acid solution. Subsequently, after dissolving the alginic acid solid at 40-60 °C, Pluronic solid is added to formulate a 30% (w/w) Pluronic solution, which is then dissolved at 0-4 °C to yield a composite continuous phase solution of alginic acid and Pluronic at the corresponding concentrations.
3. Preparation and printing of the multiphase ink system: the gelatin thermosensitive microgels are mixed with the composite continuous phase solution from Step 2 at a mass-to-volume ratio of 1 g:8 mL. After thoroughly mixing, the mixture is loaded into an extrusion printer for printing. The temperatures of both the nozzle and the receiving substrate are controlled at 32 °C. A nozzle diameter of 0.26 mm is selected, the printing speed is controlled at 10 mm/s, and the printing temperature is maintained at or above the critical temperature. After printing, the printed structure is immersed in a 3% calcium chloride solution at 37 °C (FIG. 17) for 3 minutes to optionally cross-link the alginic acid.
4. The hydrogel structure is gently soaked and washed three times with a 4 °C PBS solution for 10 minutes each time to remove excess cross-linker and uncross-linked Pluronic. Then, the hydrogel structure is immersed at a temperature above 37 °C. The oriented gelatin fibers will dissolve and flow out, forming oriented channels within the hydrogel structure, thereby achieving the fabrication of an oriented hydrogel microfiber structure based on physically cross-linked characteristic materials.
5. As described in Step 5 of Example 1, the material composition of the thermosensitive microgels can be controlled in this example, allowing for the optional sacrifice or retention of the oriented structure. When sacrifice is chosen, gelatin is used as the thermosensitive microgels. When retention is chosen, a hydrogel material that can form a stable structure through chemical or physical cross-linking is used as the main component or additive of the thermosensitive microgels. After printing at a temperature above the critical temperature and subsequent cross-linking, a hydrogel structure with oriented fine fibers inside can be obtained. In this example, methacrylated gelatin is used as the material for the thermosensitive microgels, and the resulting oriented fine fibers remain well-preserved even after 10 days in a 37 °C environment.

In the present disclosure, complex 3D structures can be obtained through a printing process that couples phase transition and deformation of the multiphase ink, enabling the fabrication of three-dimensional solid objects with personalized oriented fine fibers or fine fiber bundle structures. The manufacturing of three-dimensional oriented micron-scale hydrogel structures, oriented channel structures, and separated fibers or fiber bundles can be achieved through selective cross-linking. Additionally, by integrating with 3D bioprinting, it is possible to fabricate cell-laden three-dimensional structures with oriented features under cell-loaded conditions.

What is claimed is:

1. A 3D printing method for forming oriented fiber structures in situ, comprising the following steps:
1) preparing a reversible thermosensitive material A into microparticles/microgels and mixing it as a discrete phase with a continuous phase material B solution to obtain a multiphase printing ink;
2) loading the multiphase printing ink into an extrusion-based printer, controlling a temperature to thermally soften the discrete phase microparticles/microgels into a sol state, while extruding ink from a hopper during an extrusion printing process, wherein the discrete phase is stretched and deformed into fine fibers during extrusion process, resulting in a discrete-continuous phase composite oriented fiber structure, and implementing 3d printed structure formation based on a pre-designed three-dimensional model;
3) after printing is completed, performing optional and independent post-treatment cross-linking of a continuous phase and the discrete phase in a printed structure to obtain different three-dimensional structures with oriented characteristics.

2. The method according to claim 1, wherein in step 1), the reversible thermosensitive material A is at least one of the following materials 1)-3): 1) gelatin or modified gelatin or a mixture thereof; 2) a mixture of gelatin and a blending phase; a mixture of modified gelatin and a blending phase; a mixture of gelatin, modified gelatin, and a blending phase, wherein the blending phase is one or more of hyaluronic acid, silk fibroin, heparin, polyethylene glycol, dextran, chondroitin sulfate, sodium alginate, chitosan, and fibrinogen; 3) poly(n-isopropylacrylamide) and its modified products.

3. The method according to claim 1, wherein in step 1), the reversible thermosensitive material A also comprises a hydrogel material that forms a stable structure through chemical or physical cross-linking, and the hydrogel material that forms the stable structure through chemical or physical cross-linking serves as a main component or an additive of the discrete phase microparticles/microgels.

4. The method according to claim 1, wherein first preparing the reversible thermosensitive material A into a reversible thermosensitive material A solution, and then forming the microparticles/microgels by any of following methods: droplet microfluidics, an oil-in-water emulsion method, a complex coacervation method, an electrospraying method, or a mechanical fragmentation method;
wherein a mass concentration of the reversible thermosensitive material A in the reversible thermosensitive material A solution is 2-20%;
using a 0.9% sodium chloride solution, a phosphate-buffered saline solution, or ultrapure water as solvents to prepare the reversible thermosensitive material A solution;
the microparticles/microgels are in the form of spheres, polyhedrons, or other irregular three-dimensional shapes.

5. The method according to claim 1, wherein the discrete phase microparticles/microgels are modularly combined as components of the multiphase ink as needed, such as at least one of the following 1)-4): 1) combinations of microparticles/microgels with different sizes; 2) combinations of microparticles/microgels with different shapes; 3) combinations of microparticles/microgels with different concentrations/components of thermosensitive materials; 4) combinations of microparticles/microgels with thermosensitive properties and microparticles/microgels without thermosensitive properties.

6. The method according to claim 1, wherein a continuous phase material B maintains extrudability within an operating temperature range and has poor mutual solubility with the reversible thermosensitive material A.

7. The method according to claim 5, wherein a continuous phase material B is selected from at least one of the following 1)-3): 1) pluronic or modified pluronic or a mixture thereof; 2) a mixture of pluronic and a blending phase; a mixture of modified pluronic and a blending phase; a mixture of pluronic, modified pluronic, and a blending phase, wherein the blending phase is one or more of hyaluronic acid, silk fibroin, heparin, polyethylene glycol, dextran, chondroitin sulfate, sodium alginate, chitosan, and fibrinogen; 3) polyethylene glycol or modified polyethylene glycol or a mixture thereof.

8. The method according to claim 5, wherein when a continuous phase material B is pluronic or modified pluronic or a mixture thereof, a mass concentration of pluronic or modified pluronic or the mixture thereof in a solution of the continuous phase material B is 10-50%;
when the continuous phase material B is polyethylene glycol or modified polyethylene glycol or a mixture thereof, a mass concentration of polyethylene glycol or modified polyethylene glycol or the mixture thereof in the solution of the continuous phase material B is 5-50%;
the solution of the continuous phase material B also comprises a photoinitiator, and a mass concentration of the photoinitiator is 0.05-0.5%.

9. The method according to claim 1, wherein a mass-to-volume mixing ratio range of the discrete phase to the continuous phase material B solution is from 1 g:2 ml to 1 g:128 ml;
the temperature control involves controlling the ink hopper or a nozzle above a critical temperature, which is a sol temperature/melting temperature of the material A or higher;
a nozzle diameter is 0.2-2 mm, and a printing speed is controlled at 5-20 mm/s.

10. The method according to claim 1, wherein an operation in step 3) is: performing post-cross-linking treatment on the continuous phase in the printed structure to cross-link a hydrogel material that forms a stable structure through chemical or physical cross-linking contained in the microparticles/microgels of the reversible thermosensitive material A, obtaining a hydrogel structure with internal oriented fibers;
or performing post-cross-linking treatment on the continuous phase in the printed structure and then soaking the hydrogel structure after post-cross-linking treatment of the continuous phase above a dissolution temperature of the reversible thermosensitive material A to cause the reversible thermosensitive material A to dissolve and leach out, forming oriented channels inside the hydrogel structure and obtaining an oriented channel-embedded hydrogel structure;
or performing optional cross-linking of the continuous phase in the printed structure and the hydrogel material that forms the stable structure through chemical or physical cross-linking contained in the microparticles/microgels of the reversible thermosensitive material A to obtain a separated fiber bundle or oriented fiber scaffold.

11. A three-dimensional structure with micron-scale oriented characteristics prepared by the method according to claim 1-any wherein the three-dimensional structure with the micron-scale oriented characteristics is generated during a printing process wherein phase transition and deformation of the multiphase ink are synchronously coupled.

12. The three-dimensional structure with the micron-scale oriented characteristics according to claim 11, wherein in step 1) of the 3D printing method, the reversible thermosensitive material A is at least one of the following materials 1)-3): 1) gelatin or modified gelatin or a mixture thereof; 2) a mixture of gelatin and a blending phase; a mixture of modified gelatin and a blending phase; a mixture of gelatin, modified gelatin, and a blending phase, wherein the blending phase is one or more of hyaluronic acid, silk fibroin, heparin, polyethylene glycol, dextran, chondroitin sulfate, sodium alginate, chitosan, and fibrinogen; 3) poly(n-isopropylacrylamide) and its modified products.

13. The three-dimensional structure with the micron-scale oriented characteristics according to claim 11, wherein in step 1) of the 3D printing method, the reversible thermosensitive material A also comprises a hydrogel material that forms a stable structure through chemical or physical cross-linking, and the hydrogel material that forms the stable structure through chemical or physical cross-linking serves as a main component or an additive of the discrete phase microparticles/microgels.

14. The three-dimensional structure with the micron-scale oriented characteristics according to claim 11, wherein in the 3D printing method, first preparing the reversible thermosensitive material A into a reversible thermosensitive material A solution, and then forming the microparticles/microgels by any of following methods: droplet microfluidics, an oil-in-water emulsion method, a complex coacervation method, an electrospraying method, or a mechanical fragmentation method;
wherein a mass concentration of the reversible thermosensitive material A in the reversible thermosensitive material A solution is 2-20%;
using a 0.9% sodium chloride solution, a phosphate-buffered saline solution, or ultrapure water as solvents to prepare the reversible thermosensitive material A solution;
the microparticles/microgels are in the form of spheres, polyhedrons, or other irregular three-dimensional shapes.

15. The three-dimensional structure with the micron-scale oriented characteristics according to claim 11, wherein in the 3D printing method, the discrete phase microparticles/microgels are modularly combined as components of the multiphase ink as needed, such as at least one of the following 1)-4): 1) combinations of microparticles/microgels with different sizes; 2) combinations of microparticles/microgels with different shapes; 3) combinations of microparticles/microgels with different concentrations/components of thermosensitive materials; 4) combinations of microparticles/microgels with thermosensitive properties and microparticles/microgels without thermosensitive properties.

16. The three-dimensional structure with the micron-scale oriented characteristics according to claim 11, wherein in the 3D printing method, a continuous phase material B maintains extrudability within an operating temperature range and has poor mutual solubility with the reversible thermosensitive material A.

17. The three-dimensional structure with the micron-scale oriented characteristics according to claim 15, wherein in the 3D printing method, a continuous phase material B is selected from at least one of the following 1)-3): 1) pluronic or modified pluronic or a mixture thereof; 2) a mixture of pluronic and a blending phase; a mixture of modified pluronic and a blending phase; a mixture of pluronic, modified pluronic, and a blending phase, wherein the blending phase is one or more of hyaluronic acid, silk fibroin, heparin, polyethylene glycol, dextran, chondroitin sulfate, sodium alginate, chitosan, and fibrinogen; 3) polyethylene glycol or modified polyethylene glycol or a mixture thereof.

18. The three-dimensional structure with the micron-scale oriented characteristics according to claim 15, wherein in the 3D printing method, when a continuous phase material B is pluronic or modified pluronic or a mixture thereof, a mass concentration of pluronic or modified pluronic or the mixture thereof in a solution of the continuous phase material B is 10-50%;
when the continuous phase material B is polyethylene glycol or modified polyethylene glycol or a mixture thereof, a mass concentration of polyethylene glycol or modified polyethylene glycol or the mixture thereof in the solution of the continuous phase material B is 5-50%;
the solution of the continuous phase material B also comprises a photoinitiator, and a mass concentration of the photoinitiator is 0.05-0.5%.

19. The three-dimensional structure with the micron-scale oriented characteristics according to claim 11, wherein in the 3D printing method, a mass-to-volume mixing ratio range of the discrete phase to the continuous phase material B solution is from 1 g:2 ml to 1 g:128 ml;
the temperature control involves controlling the ink hopper or a nozzle above a critical temperature, which is a sol temperature/melting temperature of the material A or higher;
a nozzle diameter is 0.2-2 mm, and a printing speed is controlled at 5-20 mm/s.
